(19) United States
(12) Patent Application Publication
(10) Pub. No.: US 2026/0061205 A1
(43) Pub. Date: Mar. 5, 2026

(54) WIRELESS NEUROMODULATION SYSTEMS

(71) Applicant: BLUEWIND MEDICAL LTD., Herzlia (IL)

(72) Inventors: Oren ROSENBERG, Kfar Saba (IL); Michael KHMELEV, Ramla (IL); Moshe BEN CHORIN, Rehovot (IL); Guri ORON, Tel Aviv (IL)

(73) Assignee: BLUEWIND MEDICAL LTD., Herzlia (IL)

(21) Appl. No.: 19/316,383

(22) Filed: Sep. 2, 2025

Related U.S. Application Data
(60) Provisional application No. 63/690,472, filed on Sep. 4, 2024.

(51) Int. Cl. A61N 1/378; A61N 1/36

(52) U.S. Cl. CPC A61N 1/3787; A61N 1/36125; A61N 1/36167

(57) ABSTRACT

An electrostimulator implant includes (A) one or more electrodes, (B) an electrode driver, (C) an antenna circuit including: (i) an antenna receiving coil, and (ii) an antenna tuning circuit, which (a) is connected with the antenna receiving coil, (b) includes a plurality of capacitors, and (c) has a variable capacitance that provides the antenna circuit with an adjustable resonance frequency, (D) a power unit, and (E) an application-specific integrated circuit (ASIC), which (a) includes a first portion of the antenna tuning circuit, including a first subset of the capacitors, and (b) tunes the antenna circuit by setting the capacitance of the antenna circuit and controls the electrode driver to drive the electrodes. A second portion of the antenna tuning circuit is off the ASIC, and includes a second subset of the capacitors, the first and the second subsets being mutually exclusive. Other applications are also described.

### CROSS REFERENCE TO RELATED APPLICATIONS

The present application claims the priority of U.S. 63/690,472 to Rosenberg et al., filed Sep. 4, 2024 entitled, “Wireless neuromodulation systems,” which is incorporated herein by reference.

### FIELD OF THE APPLICATION

Applications of the present invention relate to transmitting power and data to an implanted medical device.

### BACKGROUND OF THE APPLICATION

Electrical power can be transferred to a medical implant by magnetic induction. A current flowing through a coil produces a magnetic field, which, in turn, will induce a current in a second coil. A coil inside a medical implant can therefore act as a receiving coil, while a coil outside a patient's body can act as a transmitting coil. A current can be driven through the transmitting coil in order to induce an induced current in the receiving coil, thereby powering the medical implant.

U.S. Pat. No. 11,213,685 to Oron et al. which is incorporated herein by reference, describes apparatus for use with a medical implant having a receiving coil. A flexible housing to be placed against skin of a subject includes a flexible transmitting coil and control circuitry for driving a current through the transmitting coil to induce a current in the receiving coil. A sensor coupled to the circuitry determines divergence of a resonance frequency of the transmitting coil when flexed from a nominal resonance frequency of the transmitting coil, occurring in the absence of any forces applied to the transmitting coil. One or more electrical components coupled to the circuitry tune the resonance frequency of the transmitting coil. A switch is coupled to each of the electrical components, the switches including transistors having capacitances that depend on the voltage applied to each switch. The circuitry applies a voltage of 30-300 volts to each switch. Other applications are also described.

US Patent Application Publication 2023/0170138 to Oron et al., which is incorporated herein by reference, describes a housing placeable against skin of a subject that includes a transmitting coil. Battery-powered control circuitry including a power stage transmits power to an implant by activating the power stage to drive a current through the transmitting coil to induce an induced current in a receiving coil of the implant. A sensor indicates divergence of a real-time resonance frequency of the transmitting coil with respect to a reference resonance frequency of the transmitting coil at which (a) efficiency of the power stage is at least 70% of a maximum efficiency as a function of the resonance frequency, and (b) the current driven through the transmitting coil is less than 96% of a maximum current drivable through the transmitting coil as a function of the resonance frequency. The circuitry reduces the divergence by tuning the resonance frequency of the transmitting coil. Other applications are also described.

### SUMMARY OF THE INVENTION

In accordance with some applications of the present invention, an electrostimulation system is provided that comprises an electrostimulator implant, which is implantable in a subject, and an external control unit (ECU), which is configured to be coupled to an external surface of the subject. By way of example and not limitation, the electrostimulator implant may comprise an implantable tibial nerve stimulator. The electrostimulator implant comprises an antenna receiving coil, one or more electrodes, and implant circuitry. Typically, the electrostimulator implant does not comprise an internal power source and is instead powered and controlled by the ECU. The implant includes an implant antenna receiving coil that receives the wireless power transfer from the ECU, and uses the power to inject a current pulse into the surrounding tissue, for example to stimulate a nerve, e.g., the tibial nerve, such as for treating overactive bladder (OAB).

A microcontroller having firmware to control the electrostimulator implant operation may have high power demand while operating (i.e., while not in a low power consumption sleep mode). This is because the microcontroller's internal RAM, multiple input/output ports, conversion units, timers and other internal units all require power for their operation. Alternatively, one or more logic state machines may control the implant operation and typically have a typically lower power demand. A logic state machine composed of logic gates and registers may create a pre-defined sequence of control signals that may be used to control the electrostimulator implant. However, a logic state machine is not as flexible as a microcontroller running a firmware. Thus, some applications of the present invention provide a combination of a microcontroller configuring one or more logic state machines that perform the control actions, and thereby benefit from both flexibility and reduced power consumption, since most of the control is performed by the logic and the microcontroller is only used for configuration. Moreover, in an implant powered by a series of RF pulses (as further described hereinbelow), configuration by the microcontroller may be performed only once (or for a limited set of pulses) and then the state machine logic may perform the control actions thus further reducing the power demand. A combination of a microcontroller and one or more logic state machines may be implemented using programable logic devices or arrays (e.g., CPLD or FPGA) or using an application specific integrated circuit (ASIC).

There is therefore provided, in accordance with some applications of the present invention, an electrostimulator implant, which is implantable in a subject and for use with an external transmitting coil, the electrostimulator implant including:
one or more electrodes;
an electrode driver, which is coupled electrically to the one or more electrodes;
an antenna circuit, which includes: (i) an antenna receiving coil; and (ii) an antenna tuning circuit, which is connected to the antenna receiving coil, and is configured to have a variable capacitance that provides the antenna circuit with an adjustable resonance frequency;
a power unit, which includes circuitry configured to convert an AC voltage induced in the antenna receiving coil to one or more DC voltages; and
digital circuitry, which includes:
   a microcontroller, which (a) includes memory, a processor core, and input/output (I/O) ports, (b) is powered by the power unit, and (c) is configured to control the electrode driver to drive the one or more electrodes to apply electrical stimulation to tissue of the subject; and
   a logic state machine, which is distinct from the microcontroller, and which is configured to tune the antenna circuit by setting the capacitance of the antenna tuning circuit.

For some applications:
the antenna tuning circuit includes a plurality of capacitors, at least some of which are switchable capacitors, and
the variable capacitance is provided by selective inclusion or exclusion of each of the switchable capacitors in the antenna tuning circuit.

For some applications, the digital circuitry includes an application-specific integrated circuit (ASIC), which comprises the microcontroller and the logic state machine.

For some applications, the ASIC includes at least a portion of the antenna tuning circuit.

For some applications, the ASIC includes an entirety of the antenna tuning circuit.

For some applications:
the antenna tuning circuit includes a plurality of capacitors, at least some of which are switchable capacitors, and
the variable capacitance is provided by selective inclusion or exclusion of each of the switchable capacitors in the antenna tuning circuit.

For some applications:
the ASIC includes a first portion of the antenna tuning circuit, which includes a first subset of the capacitors, and
a second portion of the antenna tuning circuit is off the ASIC, and includes a second subset of the capacitors, wherein the first and the second subsets are mutually exclusive.

For some applications, the first subset of the capacitors includes all of the switchable capacitors of the antenna tuning circuit.

For some applications:
the first subset of the capacitors includes some of the switchable capacitors of the antenna tuning circuit, and
the second subset of the capacitors includes some of the switchable capacitors of the antenna tuning circuit.

For some applications, an average capacitance of the capacitors of the second subset is greater than an average capacitance of the capacitors of the first subset.

For some applications, the electrostimulator implant does not include a battery.

For some applications, the microcontroller is configured to control the electrode diver to drive the one or more electrodes to apply every instance of the electrical stimulation to the tissue 0.1-5 ms after conversion of the AC voltage to the one or more DC voltages by the power unit.

For some applications:
wherein the logic state machine is configured to tune the antenna circuit by setting the capacitance of the antenna tuning circuit during a start-up phase of operation of the electrostimulator implant, and
wherein the microcontroller is configured to tune the antenna circuit by setting the capacitance of the antenna tuning circuit during an operational phase of the microcontroller after the start-up phase.

There is further provided an electrostimulator system including the electrostimulator implant, the electrostimulator system further including an external control unit, which includes the external transmitting coil and an external-control-unit power unit configured to transmit power to the electrostimulator implant by providing a current to the external transmitting coil. For example, the external-control-unit power unit may be configured to drive the external transmitting coil at a single fixed frequency.

There is further provided, in accordance with some applications of the present invention, an electrostimulator implant, which is implantable in a subject for and use with an external transmitting coil, the electrostimulator implant including:
one or more electrodes;
an electrode driver, which is coupled electrically to the one or more electrodes;
an antenna circuit, which includes: (i) an antenna receiving coil; and (ii) an antenna tuning circuit, which (a) is connected with the antenna receiving coil, (b) includes a plurality of capacitors, at least some of which are switchable capacitors, and (c) is configured to have a variable capacitance that provides the antenna circuit with an adjustable resonance frequency, the capacitance varied by selective inclusion or exclusion of each of the switchable capacitors in the antenna tuning circuit;
a power unit, which includes circuitry configured to convert an AC voltage induced in the antenna receiving coil to one or more DC voltages; and
an application-specific integrated circuit (ASIC), which (a) includes a first portion of the antenna tuning circuit, which includes a first subset of the capacitors, and (b) is configured to:
   tune the antenna circuit by setting the capacitance of the antenna circuit, and
   control the electrode driver to drive the one or more electrodes to apply electrical stimulation to tissue of the subject, and
wherein a second portion of the antenna tuning circuit is off the ASIC, and includes a second subset of the capacitors, wherein the first and the second subsets are mutually exclusive.

For some applications, the first subset of the capacitors includes all of the switchable capacitors of the antenna tuning circuit.

For some applications, the first subset of the capacitors includes some of the switchable capacitors of the antenna tuning circuit, and wherein the second subset of the capacitors includes some of the switchable capacitors of the antenna tuning circuit.

For some applications, an average capacitance of the capacitors of the second subset is greater than an average capacitance of the capacitors of the first subset.

For some applications, the ASIC includes a logic state machine, which is configured to tune the antenna circuit by setting the capacitance of the antenna tuning circuit.

For some applications, the ASIC includes a microcontroller, which (a) includes memory, a processor core, and input/output (I/O) ports, (b) is powered by the power unit, and (c) is configured to tune the antenna circuit by setting the capacitance of the antenna tuning circuit.

For some applications, the electrostimulator implant does not include a battery.

For some applications, the ASIC is configured to control the electrode driver to drive the one or more electrodes to apply every instance of the electrical stimulation to the tissue 0.1-5 ms after conversion of the AC voltage to the one or more DC voltages by the power unit.

There is further provided an electrostimulator system including the electrostimulator implant, the electrostimulator system further including an external control unit, which includes the external transmitting coil and an external-control-unit power unit configured to transmit power to the electrostimulator implant by providing a current to the external transmitting coil. For example, the external-control-unit power unit may be configured to drive the external transmitting coil at a single fixed frequency.

There is further provided, in accordance with some applications of the present invention, an electrostimulator implant, which is implantable in a subject and for use with an external transmitting coil, the electrostimulator implant including:
one or more electrodes;
an antenna circuit, which includes: (i) an antenna receiving coil; and (ii) an antenna tuning circuit, which is connected with the antenna receiving coil, and is configured to have a variable capacitance that provides the antenna circuit with an adjustable resonance frequency;
a power unit, which includes circuitry configured to convert an AC voltage induced in the antenna receiving coil to one or more DC voltages;
a logic state machine, which is powered by the power unit;
an electrode driver, which is coupled electrically to the one or more electrodes; and
a microcontroller, which (a) is distinct from the logic state machine (b) includes memory, a processor core, and input/output (I/O) ports, (c) is powered by the power unit, and (d) is configured to control the electrode driver to drive the one or more electrodes to apply electrical stimulation to tissue of the subject,
wherein the logic state machine is configured to tune the antenna circuit by setting the capacitance of the antenna tuning circuit during a start-up phase of operation of the electrostimulator implant, and
wherein the microcontroller is configured to tune the antenna circuit by setting the capacitance of the antenna tuning circuit during an operational phase of operation of the electrostimulator implant after the start-up phase.

For some applications, the electrostimulator implant includes an application-specific integrated circuit (ASIC), which comprises the logic state machine and the microcontroller.

For some applications, the electrostimulator implant includes a non-volatile memory, in which a default parameter is stored, and wherein the logic state machine is configured to tune the antenna circuit by initially setting the capacitance of the antenna tuning circuit based on the default parameter.

For some applications, the non-volatile memory includes a programmable read-only memory (PROM).

For some applications, the electrostimulator implant is configured to derive the default parameter in response to calibration of the antenna tuning circuit during production of the electrostimulator implant.

For some applications, the microcontroller is configured to tune the antenna circuit more accurately than is the logic state machine.

For some applications:
the antenna tuning circuit includes a plurality of capacitors, at least some of which are switchable capacitors, and
the variable capacitance is provided by selective inclusion or exclusion of each of the switchable capacitors in the antenna tuning circuit.

For some applications, the electrostimulator implant does not include a battery.

For some applications, the microcontroller is configured to control the electrode driver to drive the one or more electrodes to apply every instance of the electrical stimulation to the tissue 0.1-5 ms after conversion of the AC voltage to the one or more DC voltages by the power unit.

There is further provided an electrostimulator system including the electrostimulator implant, the electrostimulator system further including an external control unit, which comprises the external transmitting coil and an external-control-unit power unit configured to transmit power to the electrostimulator implant by providing a current to the external transmitting coil.

For some applications, the external-control-unit power unit may be configured to:
transmit the power to the electrostimulator implant at an initial high strength upon commencement of the start-up phase of operation of the electrostimulator implant, in order to provide sufficient energy for the logic state machine prior to tuning of the antenna circuit by the logic state machine, and
subsequently reduce the strength of the transmitted power.

For some applications, the external-control-unit power unit is configured to drive the external transmitting coil at a single fixed frequency.

There is further provided, in accordance with some applications of the present invention, an electrostimulator implant, which is implantable in a subject and for use with an external transmitting coil, the electrostimulator implant including:
one or more electrodes;
an electrode driver, which is coupled electrically to the one or more electrodes;
an antenna circuit, which includes: an (i) antenna receiving coil; and (ii) an antenna tuning circuit, which is connected with the antenna receiving coil, and is configured to have a variable capacitance that provides the antenna circuit with an adjustable resonance frequency;
a power unit, which includes circuitry configured to convert an AC voltage induced in the antenna receiving coil to one or more DC voltages; and
digital circuitry, which includes:
   a microcontroller, which (a) includes memory, a processor core, and input/output (I/O) ports, (b) is powered by the power unit, and (c) is configured to:
      control the electrode driver to drive the one or more electrodes to apply electrical stimulation to tissue of the subject during a plurality of stimulation periods, including an initial stimulation period and subsequent stimulation periods after the initial stimulation period, and
      perform non-stimulation operations during a plurality of non-stimulation periods interspersed with the stimulation periods, the non-stimulation periods including an initial non-stimulation period and subsequent non-stimulation periods after the initial non-stimulation period, the non-stimulation operations requiring less power than the electrical stimulation; and
   a register, which is configured to store a stimulation-capacitance-initiation value,
wherein the electrostimulator implant is configured to tune the antenna circuit for each of the stimulation periods by setting the capacitance of the antenna tuning circuit, including, upon commencement of each of the subsequent stimulation periods, setting an initial value of the capacitance based on the stimulation-capacitance-initiation value stored in the register.

For some applications, the electrostimulator implant is configured to determine the stimulation-capacitance-initiation value based on the capacitance of the antenna tuning circuit as tuned for one or more of the stimulation periods, and to store the determined stimulation-capacitance-initiation value in the register.

For some applications:
the one or more of the stimulation periods include the initial stimulation period, and
the electrostimulator implant is configured to determine the stimulation-capacitance-initiation value based on the capacitance of the antenna as tuned for the one or more of the stimulation periods, including the initial stimulation period, and to store the determined stimulation-capacitance-initiation value in the register.

For some applications:
the one or more of the stimulation periods include only the initial stimulation period, and
the electrostimulator implant is configured to determine the stimulation-capacitance-initiation value based on the capacitance of the antenna as tuned for the initial stimulation period, and to store the determined stimulation-capacitance-initiation value in the register.

For some applications, the stimulation-capacitance-initiation value is preconfigured and stored in the register prior to implantation of the electrostimulator implant.

For some applications:
the register is a first register,
the digital circuitry further comprises a second register, which is configured to store a non-stimulation-capacitance-initiation value, and
the electrostimulator implant is configured to tune the antenna circuit for the non-stimulation periods by setting the capacitance of the antenna tuning circuit, including, upon commencement of each of the subsequent non-stimulation periods, setting an initial value of the capacitance based on the non-stimulation-capacitance-initiation value stored in the second register.

For some applications:
the non-stimulation operations include decoding data encoded in a power signal received by the antenna circuit,
the non-stimulation periods include data-decoding periods, and
the microcontroller is configured to perform the decoding during the data-decoding periods.

For some applications, the electrostimulator implant includes an application-specific integrated circuit (ASIC), which is configured to tune the antenna circuit by setting the capacitance of the antenna tuning circuit.

For some applications, the microcontroller is configured to tune the antenna circuit by setting the capacitance of the antenna tuning circuit.

For some applications, the electrostimulator implant comprises a tuning logic state machine, which is configured to tune the antenna circuit by setting the capacitance of the antenna tuning circuit.

For some applications:
the antenna tuning circuit includes a plurality of capacitors, at least some of which are switchable capacitors, and
the variable capacitance is provided by selective inclusion or exclusion of each of the switchable capacitors in the antenna tuning circuit.

For some applications, the electrostimulator implant does not include a battery.

For some applications, the microcontroller is configured to control the electrode driver to drive the one or more electrodes to apply every instance of the electrical stimulation to the tissue 0.1-5 ms after conversion of the AC voltage to the one or more DC voltages by the power unit.

There is further provided an electrostimulator system including the electrostimulator implant, the electrostimulator system further including an external control unit, which includes the external transmitting coil and an external-control-unit power unit configured to transmit power to the electrostimulator implant by providing a current to the external transmitting coil.

For some applications, the external-control-unit power unit is configured to transmit the power to the electrostimulator implant at a higher strength during the stimulation periods than during the non-stimulation periods.

For some applications, the external-control-unit power unit is configured to drive the external transmitting coil at a single fixed frequency during the stimulation periods and the non-stimulation periods.

There is further provided, in accordance with some applications of the present invention, an electrostimulator implant, which is implantable in a subject and for use with an external transmitting coil, the electrostimulator implant including:
one or more electrodes;
an antenna circuit, which includes an antenna receiving coil;
a power unit, which includes circuitry configured to convert an AC voltage induced in the antenna receiving coil to one or more DC voltages; and
an electrode driver, which (a) includes a current source comprising a plurality of separately-activatable current sources that are arranged to provide a combined current, and (b) is configured to activate one or more of the plurality of separately-activatable current sources to drive the one or more electrodes to apply electrical stimulation to tissue of the subject at an amplitude based on which of the separately-activatable current sources are activated.

For some applications, the plurality of separately-activatable current sources are configured to provide respective currents at a same, common amplitude.

For some applications, the plurality of separately-activatable current sources are controlled by a single reference signal common to all the current sources.

For some applications, the electrode driver is configured to:
set a full-scale level of the amplitude of the electrical stimulation by setting an amplitude of the reference signal, and
fine-tune the level of the amplitude of the electrical stimulation by activating a subset of the current sources.

For some applications, the current source is a first current source and the electrode driver further comprises a second current source comprising a plurality of separately-activatable current sources, wherein the first current source and the second current source are arranged such that the electrode driver can drive the one or more electrodes to apply electrical stimulation to tissue of the subject in two polarities.

There is further provided an electrostimulator system including the electrostimulator implant, the electrostimulator system further including an external control unit, which includes the external transmitting coil and an external-control-unit power unit configured to transmit power to the electrostimulator implant by providing a current to the external transmitting coil. For example, the external-control-unit power unit may be configured to drive the external transmitting coil at a single fixed frequency.

The present invention will be more fully understood from the following detailed description of applications thereof, taken together with the drawings, in which:

### BRIEF DESCRIPTION OF THE DRAWINGS

FIGS. 1-2 are block diagrams of an electrostimulator implant which is implantable in a subject and an external control unit (ECU), in accordance with some applications of the present invention;

FIG. 3 shows a block diagram of a tuning logic state machine and a microcontroller within the electrostimulator implant, in accordance with some applications of the present invention;

FIGS. 4A-B show a bi-phasic neurostimulation pulse that comprises three parts, in accordance with some applications of the present invention;

FIG. 5 is a block diagram of the electrostimulator implant, in accordance with some applications of the present invention;

FIGS. 6A-B depict details of a current source, in accordance with some applications of the present invention; and

FIG. 7 is a block diagram of the electrostimulator implant, in accordance with some applications of the present invention.

### DETAILED DESCRIPTION

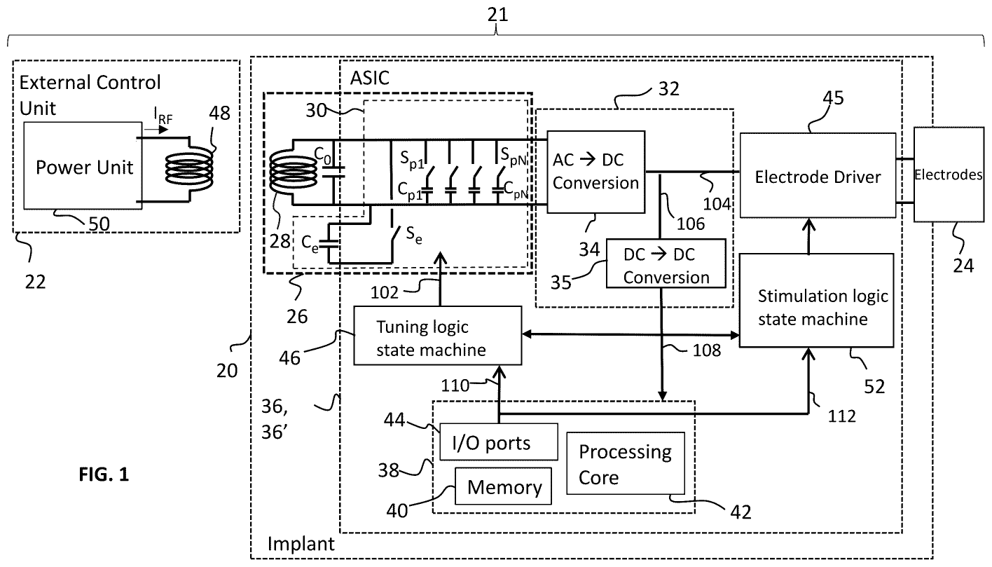

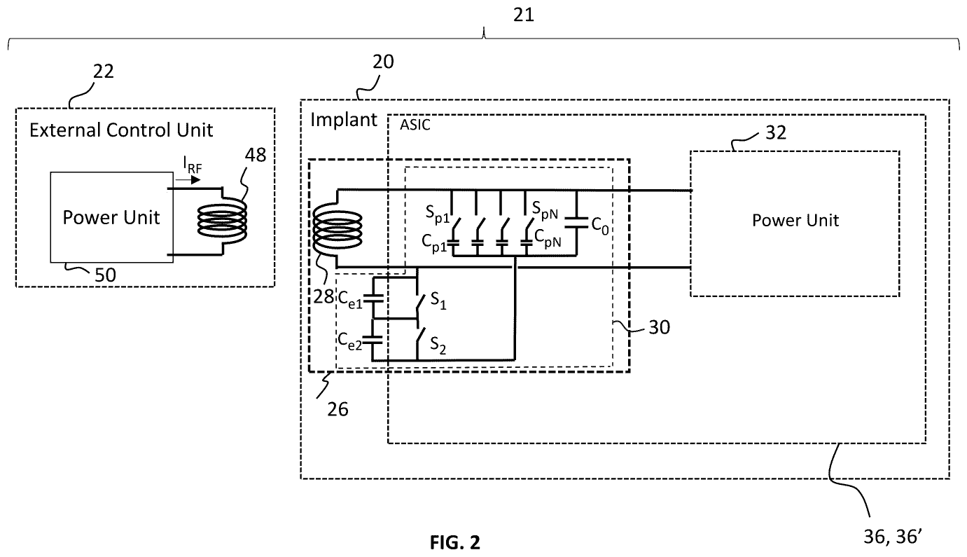

Reference is now made to FIGS. 1-2, which are block diagrams of an electrostimulator implant 20 which is implantable in a subject and an external control unit (ECU) 22, in accordance with some applications of the present invention. As described hereinabove, use of a combination of a microcontroller and one or more logic state machines to operate electrostimulator implant 20 may decrease the overall power consumption of electrostimulator implant 20 and may be implemented using programable logic devices or arrays (e.g., CPLD or FPGA) or using an application specific integrated circuit (ASIC).

Thus, for some applications, electrostimulator implant 20 includes the following:
one or more electrodes 24 for injecting a current pulse into tissue surrounding electrostimulator implant 20, for example to stimulate a nerve, e.g., the tibial nerve, such as for treating overactive bladder (OAB);
an electrode driver 45 (further described hereinbelow) which is coupled electrically to the one or more electrodes and configured to drive the one or more electrodes to apply electrical stimulation to tissue of the subject;
an antenna circuit 26, which includes: (i) an antenna receiving coil 28; and (ii) an antenna tuning circuit 30, which is connected to antenna receiving coil 28, and has a variable capacitance that provides antenna circuit 26 with an adjustable resonance frequency;
a power unit 32, which comprises AC/DC conversion circuitry 34 configured to convert an AC voltage induced in antenna receiving coil 28 to one or more DC voltages; and
digital circuitry 36, e.g., an application-specific integrated circuit (ASIC) 36′, which comprises:
   a microcontroller 38, also known as a microcontroller unit (MCU), which (a) comprises memory 40, a processor core 42, and input/output (I/O) ports 44, (b) is powered by power unit 32, and (c) is configured to control electrode driver 45 to drive the one or more electrodes 24 (e.g., by directly controlling electrode driver 45, or by configuring a stimulation logic state machine 52 which controls electrode driver 45, as further described hereinbelow) to apply electrical stimulation to tissue of the subject; and
   a tuning logic state machine 46, which is distinct from microcontroller 38, and which is configured to tune antenna circuit 26 by setting the capacitance of antenna tuning circuit 30.

Typically, electrostimulator implant 20 does not comprise a battery. Thus, electrostimulator implant 20 typically operates in connection with ECU 22, which provides power to electrostimulator implant 20 through a wireless connection. For some applications, an electrostimulator system 21 is provided, including electrostimulator implant 20 and ECU 22. ECU 22 typically has an external transmitting coil 48 and an ECU power unit 50. ECU power unit 50 transmits power to electrostimulator implant 20 by providing a current to the external transmitting coil 48, e.g., by driving a current at RF frequency (e.g. at a single fixed frequency, e.g., at 6.78 MHz) through transmitting coil 48. Typically, external transmitting coil 48 is tuned prior to transmitting power to electrostimulator implant 20.

The oscillating magnetic fields created by the current in transmitting coil 48 induce an AC voltage in antenna receiving coil 28. For some applications, power unit 32 converts the induced AC voltage to one or more DC voltages, e.g., a main DC voltage, using AC/DC conversion circuitry 34. The main DC voltage is then used to power electrostimulator implant 20, either directly (e.g. to power electrode driver 45, as indicated by line 104 in FIG. 1) or through further DC/DC conversion within power unit 32, as indicated by line 106 in FIG. 1 (e.g., for various logic state machines, as indicated by arrow 108 in FIG. 1).

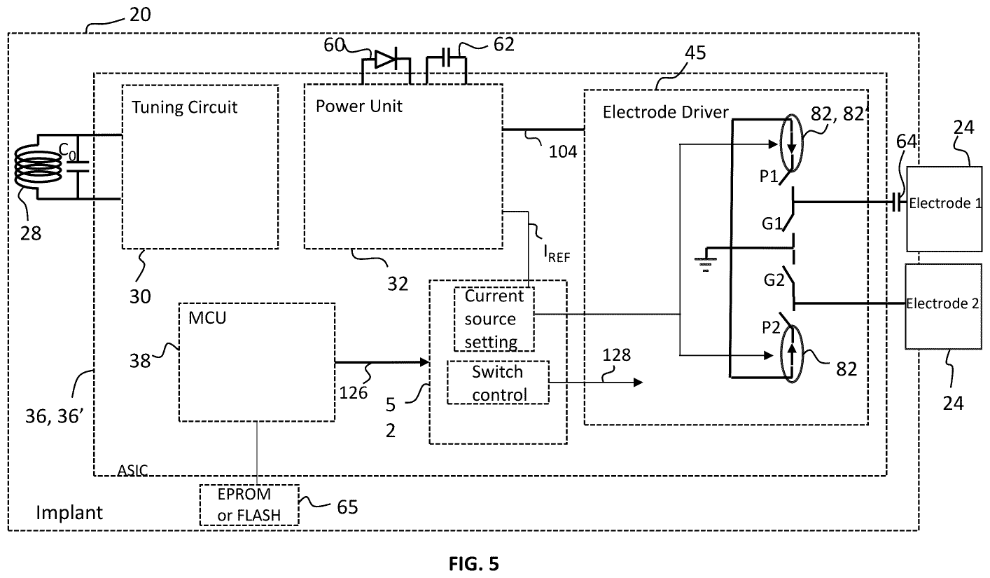

Typically, the AC/DC conversion includes rectification of the induced AC voltage, followed by ripple smoothing by capacitors (both of which may reside within ASIC 36′ or may be external to it as indicated by diode 60 and capacitor 62 in FIG. 5) and overvoltage protection circuitry that limits the main DC voltage to a maximum.

For some applications, digital circuitry 36 is implemented as ASIC 36′ and includes power unit 32. Power unit 32 may also include (a) various DC regulators 35 that convert the main DC voltage to other DC voltages for use by units within ASIC 36′ (as well as by other implant electronics units external to ASIC 36′, e.g., EPROM/FLASH memory), (b) a temperature insensitive bandgap voltage reference that is used for setting other regulator voltages and the current reference used by electrode driver 45 (further described hereinbelow), and (c) other units relating to the DC voltage and current. For some applications, having a plurality of DC regulators that may be enabled or disabled may provide means to switch off parts of electrostimulator implant 20 and, in particular, electronic circuitry of ASIC 36′, thus controlling the power consumption of electrostimulator implant 20.

Other units within ASIC 36′ may include electrode driver 45, antenna tuning circuit 30 and other analog and digital units. Electrode driver 45, as further described hereinbelow, comprises at least one, e.g., two, current sources connected to electrode(s) 24 and switches that control the connection between the current sources and the different electrodes for the different stages of the stimulation pulse. Antenna tuning circuit 30, as further described hereinbelow, includes capacitors at least some of which are switchable capacitors, allowing them to be included in or excluded from the total capacitance connected to antenna receiving coil 28.

The digital control portion of the ASIC includes microcontroller 38 and other logic state machines. The logic state machines are configurable, hard wired logic units that are responsible for performing a specific task. Two examples of this are (i) stimulation logic state machine 52 controls electrode driver 45, in particular with regard to the setting the current sources to provide the required currents and timing of the switching of the different current sources so that the required stimulation pulse is provided, and (ii) tuning logic state machine 46 controls the switchable capacitors of antenna tuning circuit 30, as indicated by arrow 102 in FIG. 1. Microcontroller 38 has processing core 42, memory 40 for storing the program and the parameters sent by ECU 22 and input and output (I/O) ports 44 used to monitor the conditions of the different elements of the circuitry and to configure the different logic state machines. For example, microcontroller 38 operates in order to configure the logic state machines (e.g., set the parameters), as indicated by arrows 110 and 112 in FIG. 1, and then microcontroller 38 goes to sleep and the logic state machines perform their specific tasks based on their set configurations. Subsequently, microcontroller 38 wakes up from sleep and operates at the beginning of each transmitted RF pulse for a few microseconds. As further described hereinbelow, if however, microcontroller 38 shuts down and loses the data stored in memory 40, then microcontroller 38 is typically re-booted and re-programmed by ECU 22, in which case it operates for a longer period of time, e.g., at least 2 and/or less than 200 ms, e.g., at least 5 and/or less than 120 ms.

For example, for some applications, microcontroller 38 is configured to control electrode driver 45 to drive one or more electrodes 24 to apply every instance of the electrical stimulation to the tissue within 5 ms, e.g., within 100 microseconds, of conversion of the AC voltage to the one or more DC voltages by power unit 32. As further described hereinbelow, this small amount of time between (a) the conversion of the induced AC voltage to the one or more DC voltages and (b) the application of the electrical stimulation to the tissue is referred to as a control period of each transmitted RF pulse.

ASIC 36′ is supported by external electronics that support its operation. These external electronic units are not incorporated into the structure of ASIC 36′ because they cannot be easily implemented there, or due to constraints on size or power dissipation. These external electronic units are accessed by ASIC 36′ through pads that enable connectivity to the external electronic units placed on the printed circuit board (PCB) of electrostimulator implant 20. Additional pads may be included for example for connecting an external voltage supply (non-wireless power source) for use during testing and calibration of the PCB.

These external electronic units include both analog units and digital units.
The external analog units may include one or more parts of antenna circuit 26 (e.g., antenna receiving coil 28, and, for some applications, one or more capacitors) that connect to antenna tuning circuit 30 on ASIC 36′, one or more rectifying diodes (e.g., rectifying diode 60) that convert the received RF power from ECU 22 to a DC voltage, and a ripple smoothing capacitor 62 (all connected to power unit 32 and illustrated in FIG. 5), and a DC blocking capacitor 64 of electrode driver 45 (shown in FIG. 5).
The external digital units include an external programable non-volatile memory (EPROM or FLASH) 65 (shown in FIG. 5) that is used to store the programs to be loaded into microcontroller 38.

### Wireless Power Transfer and Tuning

In order to provide more efficient power transfer from ECU 22 to electrostimulator implant 20, it is advantageous for the wireless power transfer to be resonant. Therefore, the antenna receiving coil 28 is coupled with one or more capacitors (e.g. C₀) to create a resonant circuit. The higher the quality factor of the resonance the higher the efficiency of the power transfer is. However, the high-quality factor also implies a narrow band width of reception (resonance). While the wireless power carrier frequency transmitted by ECU 22 may be varied, in many cases this is not allowed due to regulatory restrictions on electromagnetic emissions. For some applications, the antenna circuitry resonance frequency is fixed, requiring high accuracy of the components to obtain an accurate resonant frequency to provide efficient power transfer. Alternatively, antenna tuning circuit 30 is included as a part of implant antenna circuit 26 to better match the frequency of the received carrier, as shown in FIG. 1. Antenna tuning circuit 30 is connected to antenna circuit 26 and can alter the effective capacitance connected to antenna receiving coil 28, thus changing the antenna resonance frequency.

Having a tunable resonant frequency has several advantages. First, it allows the use of less accurate components, thus simplifying the production process of electrostimulator implant 20, reducing costs, and improving production yield (if the wireless power transmission were outside the implant antenna resonance band, a non-tunable implant would not receive enough power for operation). Moreover, antenna tuning circuit 30 enables an adjustment of the resonance frequency so that the power transfer can be optimized, i.e., best adapted for implant operation.

In this respect, it should be understood that the wireless power transfer from ECU 22 to electrostimulator implant 20 depends on their relative geometry, e.g. the relative position of antenna receiving coil 28 with respect to external transmitting coil 48, including the distance from electrostimulator implant 20 to external transmitting coil 48, and the tilt of antenna receiving coil 28 with respect to the magnetic field lines created by external transmitting coil 48. Optionally, placement of ECU 22 with respect to electrostimulator implant 20 with respect is performed using techniques described in US Patent Application Publication 2022/0355120 to Oron et al., which is incorporated herein by reference. Limits on some of these parameters (e.g. the minimum distance between electrostimulator implant 20 and external transmitting coil 48) are set during the implantation procedure, and thus affect the wireless power transfer. For example, implant 20 may be implanted deep in the tissue and thus experience relatively low magnetic fields when ECU 22 transmits power. In this case, optimal adjustment would attempt to increase the power delivered to the implant. However, when the implantation is shallow, the implant may receive high levels of power, which may result in a high DC voltage that may cause harm to internal circuitry of electrostimulator implant 20. Internal over voltage protection circuitry may clip this voltage to a maximum, but this causes heating within ASIC 36′. Alternatively, antenna tuning circuit 30 may be optimized by shifting the resonance frequency away from maximum power transfer, so that the implant DC voltage is reduced, thus enabling a controlled adjustable overvoltage protection.

### Tuning Circuitry Implementation

For some applications, antenna tuning circuit 30 comprises a plurality of capacitors Cp1 . . . CpN, at least some of which are switchable capacitors, connected (in parallel or in series or a combination of both) to a non-switchable capacitor C₀ as shown for example in FIGS. 1 and 2. Variable capacitance is provided to antenna circuit 26 by selective inclusion or exclusion of each of the switchable capacitors Cp1 . . . CpN in antenna tuning circuit 30. For some applications, the plurality of capacitors Cp1 . . . CpN (of different capacitances) are in parallel to capacitor C₀ and are switchable via respective switches Sp1 . . . SpN, so they can selectively be excluded or included within the total capacitance of antenna circuit 26. It is noted that C₀ may be external to ASIC 36′ circuitry (such as is shown in FIG. 1) or embedded within ASIC 36′ as a part of tuning circuit 30 (such as is shown in FIG. 2).

For some applications, ASIC 36′ comprises at least a portion of antenna tuning circuit 30, e.g., an entirety of antenna tuning circuit 30 (configuration not shown). Typically, capacitors Cp1 . . . CpN may be internal to ASIC 36′ and may have varying capacitance values. It is noted that C₀ may be external to ASIC 36′ circuitry (such as is shown in FIG. 1) or embedded within ASIC 36′ as a part of antenna tuning circuit 30 (such as is shown in FIG. 2). However, capacitance values that may be created within an ASIC circuitry are limited by the technology. Therefore, if the tuning requires capacitance values which cannot be produced within ASIC 36′, a capacitor or plurality of capacitors may be placed on the electronic board outside ASIC 36′ and connected to the ports of ASIC 36′. For example, a capacitor Ce may be placed outside ASIC 36′ and connected via a switch Se (within ASIC 36′) in parallel to the capacitor C₀, thus enlarging the tuning range.

An alternative combination of internal and external capacitors is shown in FIG. 2. The capacitor C₀ is on ASIC 36′ in this configuration and is connected in parallel to a plurality of switchable capacitors Cp1 . . . CpN, which are switchable via the switches Sp1 . . . SpN. Further tuning may be provided via capacitors Ce1 and Ce2 (external to ASIC 36′ but within electrostimulator implant 20) and connected via switches S1 and S2 (within ASIC 36′). If S1 and S2 are closed, the parallel combination of Co and the included capacitors from the switchable Cp1 . . . CpN (referred to hereinbelow as “the parallel combination”) is the total capacitance connected to the antenna coil (Ce1 and Ce2 are bypassed by the switches S1 and S2). If S1 is open and S2 closed, Ce2 is connected in series with the parallel combination. If S1 is closed and S2 open, Ce1 is connected in series with the parallel combination. If both S1 and S2 are open the parallel combination is connected in series with the series combination of Ce1 and Ce2.

Thus, for some applications, (i) ASIC 36′ includes a first portion of antenna tuning circuit, which includes a first subset of the capacitors (e.g., Cp1 . . . CpN, and for some applications also C₀), and (ii) a second portion of antenna tuning circuit 30 is off ASIC 36′ and includes a second subset of the capacitors (e.g., Ce, or the combination of Ce1 and Ce2, and for some applications C₀). For some applications, the first subset of the capacitors includes all of the switchable capacitors of antenna tuning circuit 30 (configuration not shown). Alternatively, for some applications, the first subset of the capacitors includes some of the switchable capacitors (e.g., Cp1 . . . CpN) of antenna tuning circuit 30, and the second subset of the capacitors includes some of the switchable capacitors of the antenna tuning circuit (e.g., Ce or the combination of Ce1 and Ce2). For some applications, an average capacitance of the capacitors of the second subset is greater than an average capacitance of the capacitors of the first subset.

The switches that control the inclusion or exclusion of the capacitors of antenna tuning circuit 30 may be implemented as FETs, e.g., MOSFETs. These FETs may be designed so that without power available they allow some of the capacitors to be either included or excluded. When ASIC 36′ is powered, the condition of each of the FETs is controlled by the signals provided by tuning logic state machine 46.

For some applications, ASIC 36′ tunes antenna circuit 26 by setting the capacitance of antenna tuning circuit 30 using either microcontroller 38 or tuning logic state machine 46. Thus, for some applications, electrostimulator implant 20 includes the following:
one or more electrodes 24;
electrode driver 45, which is coupled electrically to the one or more electrodes and configured to drive the one or more electrodes to apply electrical stimulation to tissue of the subject;
antenna circuit 26, which includes: (i) antenna receiving coil 28; and (ii) antenna tuning circuit 30, which (a) is connected with antenna receiving coil 28 (e.g., in series), (b) comprises a plurality of capacitors (Cp1 . . . CpN, and/or Ce or the combination of Ce1 and Ce2), at least some of which are switchable capacitors, and (c) is configured to have a variable capacitance that provides antenna circuit 26 with an adjustable resonance frequency, the capacitance varied by selective inclusion or exclusion of each of the switchable capacitors in antenna tuning circuit 30;
power unit 32, which comprises AC/DC conversion circuitry 34 configured to convert an AC voltage induced in antenna receiving coil 28 to one or more DC voltages; and
ASIC 36′, which (a) includes a first portion of antenna tuning circuit 30, which includes a first subset of the capacitors (as described hereinabove), and (b) is configured to:
   tune antenna circuit 26 by setting the capacitance of antenna circuit 26, and
   control electrode driver 45 to drive one or more electrodes 24 to apply electrical stimulation to tissue of the subject, and
wherein a second portion of antenna tuning circuit 30 is off ASIC 36′, and includes a second subset of the capacitors, wherein the first and the second subsets are mutually exclusive (as described hereinabove).

For some applications, ASIC 36′ includes tuning logic state machine 46, which is configured to tune antenna circuit 26 by setting the capacitance of antenna tuning circuit 30. Alternatively or additionally, ASIC 36′ includes microcontroller 38, which (a) comprises memory 40, processor core 42, and input/output (I/O) ports 44, (b) is powered by power unit 32, and (c) is configured to tune antenna circuit 26 by setting the capacitance of antenna tuning circuit 30.

As described hereinabove, for some applications, the first subset of the capacitors includes all of the switchable capacitors of antenna tuning circuit 30 (configuration not shown). Alternatively, for some applications, the first subset of the capacitors includes some of the switchable capacitors (e.g., Cp1 . . . CpN) of antenna tuning circuit 30, and the second subset of the capacitors includes some of the switchable capacitors of the antenna tuning circuit (e.g., Ce or the combination of Ce1 and Ce2). For some applications, an average capacitance of the capacitors of the second subset is greater than an average capacitance of the capacitors of the first subset.

### Pulse Shape

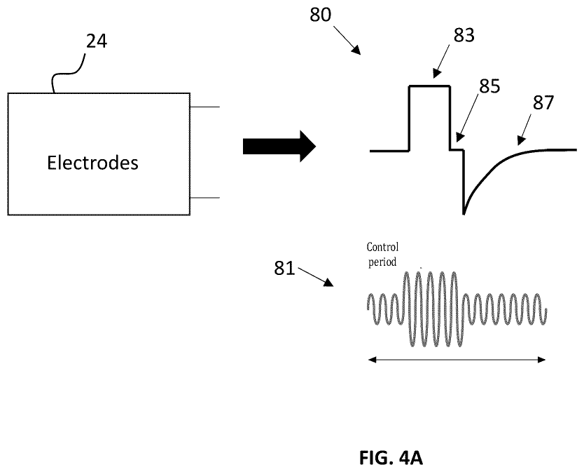

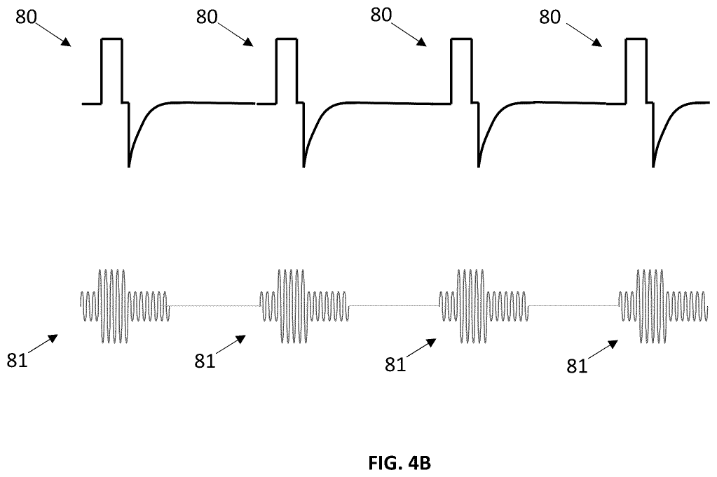

Reference is now made to FIGS. 4A-B, which show a bi-phasic neurostimulation pulse 80 that comprises three parts, in accordance with some applications of the present invention. The first part of neurostimulation pulse 80 is a rectangular constant current amplitude pulse of pre-set pulse width (“active stimulation” period 83). This is followed by the second part which is a short interphase delay period 85 of zero current and then by the third part which is an inverse polarity “discharge” period 87. FIG. 4 also shows the RF current pulse 81 from ECU 22 that powers neurostimulation pulse 80. It is noted that for each pulse, prior to the active stimulation period, there is an additional period referred to herein as a “control period” during which there is no stimulation to the tissue. During the control period, ECU 22 typically transmits power to wake up microcontroller 38, and sends data to microcontroller 38. It is noted that as used herein, including in the claims, the term “non-stimulation period” refers to time during the RF current pulse 81 in which active stimulation is not performed, e.g., to a majority of the duration of the RF current pulse in which active stimulation is not performed, i.e., the combined control period and discharge period of each neurostimulation pulse 80. FIG. 4B shows how neurostimulation pulses 80 repeat at the frequency of power delivery by ECU 22 to electrostimulator implant 20.

Neurostimulation pulses are usually bi-phasic since there is a requirement that the total DC current density injected to the tissue shall be below 0.75 microamps/mm2 (ISO 14708-1, section 16.2). However, in most cases the active injection occurs in both directions and the current balance is achieved by accurate control of the pulse shape (in both current polarities). The injection of current in both polarities requires high level power delivery during the whole pulse (both polarities). This is undesirable for an externally powered implant. Thus, for some applications of the present invention, the implant employs DC blocking capacitor 64 (FIG. 5) placed between a current source 82 (FIG. 5) and the injecting electrode 24. DC blocking capacitor 64 is charged during the active stimulation phase of the pulse and then passively discharges through the tissue during the discharge period, using only low amount of power transfer.

### Tuning Logic State Machine

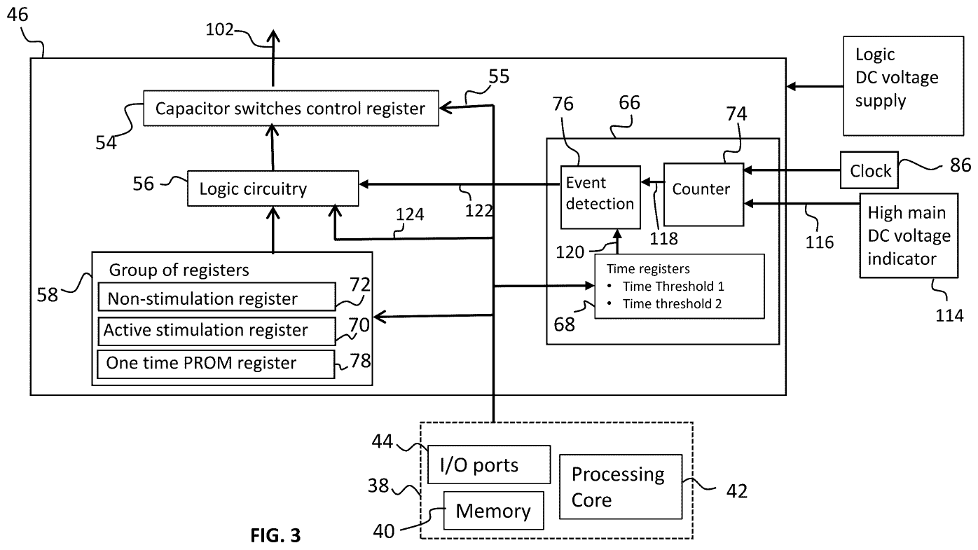

Reference is now made to FIG. 3, which shows a block diagram of tuning logic state machine 46 and microcontroller 38, in accordance with some applications of the present invention. For some applications, tuning logic state machine 46 is responsible for adjusting the state of the switches (Sp1-SpN, Se, S1, S2) so that an optimal operation condition may be achieved for antenna circuit 26. As shown in FIG. 3, the status of the different switches may be kept in a switches control register 54 that stores the digital bits representing the status of each switch (open/closed). The output of switches control register 54 may be used (directly or via an appropriate driver) to control the gate voltage of the respective FET and thus its condition (conducting/non-conducting).

The switches control register value may be set directly by microcontroller 38 (as indicated by arrow 55), or alternatively by logic circuitry 56 that reads the register values from a group 58 of switches configuration registers (e.g., a predefined PROM (programable ROM) or RAM or combination thereof) within tuning logic state machine 46 and loads the register values into switches control register 54. Group 58 may include one or more registers that can be read by logic circuitry 56 and loaded into switches control register 54 based on signals arriving from other elements within the implant circuitry, e.g., microcontroller 38, and a timer logic unit 66 (further described hereinbelow).
1. For some applications, (such as when utilizing tuning protocol B or C, described hereinbelow) at different stages during operation electrostimulator implant 20 requires different amounts of power. For example, lower power is required during the operation of the electronics when the electrical stimulation is not being applied to the tissue of the subject (non-stimulation period), and higher power is required when the electrical stimulation is being applied to the tissue (active stimulation period). Accordingly, ECU 22 may provide lower RF power during non-stimulation periods and higher RF level during the stimulation periods. Moreover, the load presented to the resonant circuit during the operation of electrostimulator implant 20 may vary. This load variation affects the resonant frequency of implant antenna circuit 26, thus affecting power transfer efficiency. One possibility is to adjust the antenna resonance under each load condition (non-stimulation or active stimulation) and, via antenna tuning circuit 30, to vary the antenna resonance between the different stages of the RF power pulse, so that power transfer is optimal at all stages (such as when utilizing tuning protocol C described hereinbelow). Another possibility is to adjust the antenna resonance as a compromise between the different load conditions (such as when utilizing tuning protocol B described hereinbelow). For example, if a certain capacitance Ceff is needed for optimal adjustment of the antenna resonance for non-stimulation and Ceff+dC is needed for optical adjustment of the antenna resonance for active stimulation, then Ceff+0.5*dC or Ceff+0.75*dC may be used as a compromise between the two conditions (dC is typically much lower than Ceff).

For some applications, ECU 22 may provide lower RF power during non-stimulation periods and higher RF level during the active stimulation periods. As a result, the main voltage derived by the implant will change as a result of the switching. This voltage change may be detected by the implant and used as a trigger (with a pre-set time delay) to the switch between the two capacitor configurations. Alternatively, the switching may be controlled by a timer measuring time events from a pre-defined zero timing (e.g. beginning of the RF pulse or MCU signal).

For some applications, the tuning control may be achieved using microcontroller 38 (such as when utilizing tuning protocol B or C described hereinbelow). For example, after microcontroller 38 boots up it may perform a scan of the possible configurations of the switches (Sp1-SpN, Se, S1, S2) or a subset of the switches configurations to explore the variation of the measured main DC voltage with the antenna resonance (as controlled by the switches configuration). Microcontroller 38 then decides, based on the measured main DC voltages, whether to adjust the configuration of the switches for maximum main DC voltage (e.g., if the measured voltage is low) or to tune the resonance away from the frequency of external transmitting coil 48 in order to limit the magnitude of the DC voltage (e.g., if high voltage is obtained at resonance match conditions).

Microcontroller 38 may perform a scan of the tuning switches (Sp1-SpN, Se, S1, S2) in conditions appropriate for operation during the non-stimulation period and for operation during active stimulation, and determine the appropriate combination of switch settings for each stage of operation (such as when utilizing tuning protocol C described hereinbelow). Microcontroller 38 may then store these combinations in respective registers 70 and 72 within group 58 of switches configuration registers within tuning logic state machine 46.

Thus, for some applications, electrostimulator implant 20 includes:
one or more electrodes 24;
electrode driver 45, which is coupled electrically to the one or more electrodes and configured to drive the one or more electrodes to apply electrical stimulation to tissue of the subject;
antenna circuit 26, which comprises: (i) antenna receiving coil 28; and (ii) antenna tuning circuit 30, which is connected to antenna receiving coil 28, and is configured to have a variable capacitance that provides antenna circuit 26 with an adjustable resonance frequency;
power unit 32, which comprises AC/DC conversion circuitry 34 configured to convert an AC voltage induced in antenna receiving coil 28 to one or more DC voltages; and
digital circuitry, which comprises:
   microcontroller 38, which (a) comprises memory 40, processor core 42, and input/output (I/O) ports 44, (b) is powered by power unit 32, and (c) is configured to:
      control electrode driver 45 to drive one or more electrodes 24 to apply electrical stimulation to tissue of the subject during a plurality of stimulation periods, including an initial stimulation period and subsequent stimulation periods after the initial stimulation period, and
      perform non-stimulation operations during a plurality of non-stimulation periods interspersed with the stimulation periods, the non-stimulation periods including an initial non-stimulation period and subsequent non-stimulation periods after the initial non-stimulation period, the non-stimulation operations requiring less power than the electrical stimulation; and
   an active stimulation register 72 (e.g., in PROM or RAM, within group 58), which is configured to store a stimulation-capacitance-initiation value (i.e., a default configuration of the tuning switches appropriate for operation during active stimulation).

Electrostimulator implant 20 is configured to tune antenna circuit 26 for each of the stimulation periods by setting the capacitance of antenna tuning circuit 30, including, upon commencement of each of the subsequent stimulation periods, setting an initial value of the capacitance based on the stimulation-capacitance-initiation value stored in register 72.

For some applications, the default configuration of the tuning switches appropriate for operation during active stimulation (such as when utilizing tuning protocol B or C described hereinbelow) is determined during operation of the device (rather than predetermined during manufacture). Thus, for some applications, electrostimulator implant 20, e.g., microcontroller 38, determines the stimulation-capacitance-initiation value based on the capacitance of the antenna tuning circuit as tuned for one or more of the stimulation periods and stores the determined stimulation-capacitance-initiation value in register 72. For some applications, the one or more stimulation periods includes the initial stimulation period and electrostimulator implant 20, e.g., microcontroller 38, determines the stimulation-capacitance-initiation value based on the capacitance of the antenna tuning circuit as tuned for the one or more of the stimulation periods, including the initial stimulation period, and stores the determined stimulation-capacitance-initiation value in register 72.

For some applications, the default configuration of the tuning switches appropriate for operation during active stimulation is determined only once during operation of the device, i.e., during the first stimulation period. Thus, for some applications the one or more of the stimulation periods include only the initial stimulation period, and electrostimulator implant 20, e.g., microcontroller 38, determines the stimulation-capacitance-initiation value based on the capacitance of the antenna as tuned for the initial stimulation period, and stores the determined stimulation-capacitance-initiation value in register 72.

For some applications, the digital circuitry (e.g., tuning logic state machine 46) further includes a second register 70 (e.g., in PROM or RAM, within group 58), which is configured to store a non-stimulation-capacitance-initiation value (i.e., a default configuration of the tuning switches appropriate for operation of electrostimulator implant 20 during the non-stimulation periods), and electrostimulator implant 20 is configured to tune antenna circuit 26 for the non-stimulation periods by setting the capacitance of antenna tuning circuit 30, including, upon commencement of each of the subsequent non-stimulation periods, setting an initial value of the capacitance based on the non-stimulation-capacitance-initiation value stored in second register 70.

For some applications, the non-stimulation operations include decoding data encoded in a power signal received by the antenna circuit 26 from ECU 22. The non-stimulation periods of electrostimulator implant 20 include data-decoding periods (e.g., as part of the “control period” as described hereinabove), during which microcontroller 38 performs decoding of the encoded data.

For some applications, electrostimulator implant 20 includes an application-specific integrated circuit (ASIC 36′), which is configured to tune antenna circuit 26 by setting the capacitance of antenna tuning circuit 30. For some applications, microcontroller 38 is configured to tune antenna circuit 26 by setting the capacitance of antenna tuning circuit 30. For some applications, electrostimulator implant 20 includes tuning logic state machine 46, which is configured to tune antenna circuit 26 by setting the capacitance of antenna tuning circuit 30.

Tuning logic state machine 46 may also include timer logic unit 66 (such as when utilizing tuning protocol C described hereinbelow) which includes time duration registers 68. Tuning logic state machine 46 can thus be set to run based on signals arriving from ASIC circuitry. For example, when a signal 114 is received indicating that a high enough voltage level is created for operation (due to RF level, e.g., at the beginning of an RF pulse sent from ECU 22) a counter 74 may count a clock ticking signal until a first (configurable) time threshold 1 is exceeded, at which stage the configuration of the capacitor switches appropriate for active stimulation is fetched by logic circuitry 56 from register 72 of group 58 and loaded into switches control register 54. Counter 74 then continues to evaluate the elapsed time until a second time threshold 2 is crossed, upon which logic circuitry 56 fetches the configuration of the capacitor switches appropriate for no stimulation from register 70 of group 58 and loads the configuration into switches control register 54. Logic circuitry 56 then waits until another signal 114 is received indicating the beginning of a new pulse, at which point counter 74 is reset, as indicated by arrow 116. Event detection unit 76 detects an event such as counter 74 being reset, as indicated by arrow 118, and the respective time thresholds 1 and 2 being reached, as indicated by arrow 120. Arrow 122 represents event detection unit 76 indicating to logic circuitry 56 that a time threshold has been reached.

The availability of tuning enables less strict accuracy of the resonant antenna circuit 26. However, without initial tuning the antenna resonance may be initially far from the RF frequency provided by the ECU 22, such that the full implant (and in particular the MCU) is not able to boot up and perform the tuning. This issue may be solved by resorting to accurate adjustment of the resonant circuitry components during production, however this solution eliminates much of the advantage of the tuning circuitry. Alternatively, at initial implant power up the power consumption of the implant may be configured to be low. As stated above, power unit 32 may have a plurality of DC regulators which may be enabled or disabled. For some applications, during a start-up phase of operation of electrostimulator implant 20, only a regulator providing power to antenna tuning circuit 30 and to tuning logic state machine 46 are operated, thus limiting the power consumption of ASIC 36′. Tuning logic state machine 46 provides initial tuning to antenna circuit 26 (further described hereinbelow). Following this initial tuning, enough power for the full ASIC operation is available and the other DC regulators may operate enabling the operation of the other ASIC elements (and in particular booting microcontroller 38). When microcontroller 38 finishes its boot sequence it provides a “microcontroller on” signal, as indicated by arrow 124, that indicates to tuning logic state machine 46 that microcontroller 38 is able to configure and set the different configurations (stored in respective registers 70 and 72) of the tune logic state machine 46.

When powered up and in the absence of the “microcontroller on” signal, the tuning logic machine loads a capacitance configuration value from a non-volatile memory 78 of group 58, e.g., a programmable read-only memory (PROM), which stores a pre-defined capacitance configuration value, into register 54. This tuning provides enough power transfer into the implant circuitry. An appropriate logic within the power unit may detect this increase of power and enable the DC regulator to provide power for the microcontroller. Upon booting up of the microcontroller, the microcontroller may further optimize the tuning and store the corresponding values in RAM registers within group 58 as discussed elsewhere. The microcontroller provides a “microcontroller on” signal that indicates to the tuning logic control machine to use the optimized stored values rather than the initial PROM value as long as power is available. The microcontroller may also enable other regulators and units within the ASIC, either prior or after the tuning optimization, and may repeat the optimization process at different stages of the operation.

Thus, for some applications, electrostimulator implant 20 includes:
one or more electrodes 24;
electrode driver 45, which is coupled electrically to the one or more electrodes and configured to drive the one or more electrodes to apply electrical stimulation to tissue of the subject;
antenna circuit 26, which includes: (i) antenna receiving coil 28; and (ii) antenna tuning circuit 30, which is connected with antenna receiving coil 28, and is configured to have a variable capacitance that provides antenna circuit 26 with an adjustable resonance frequency;
power unit 32, which includes AC/DC conversion circuitry 34 configured to convert an AC voltage induced in antenna receiving coil 28 to one or more DC voltages;
a logic state machine, e.g., tuning logic state machine 46, which is powered by power unit 32; and
microcontroller 38, which (a) is distinct from the logic state machine, (b) includes memory 40, processor core 42, and input/output (I/O) ports 44, (c) is powered by power unit 32, and (d) is configured to control electrode driver 45 to drive one or more electrodes 24 to apply electrical stimulation to tissue of the subject,
   wherein the logic state machine, e.g., tuning logic state machine 46, is configured to tune antenna circuit 26 by setting the capacitance of antenna tuning circuit 30 during a start-up phase of operation of electrostimulator implant 20, and
   wherein microcontroller 38 is configured to tune antenna circuit 26 by setting the capacitance of antenna tuning circuit 30 during an operational phase of operation of microcontroller 38 after the start-up phase.

The start-up phase is defined as the time from when electrostimulator implant 20 starts to get power from ECU 22 until enough power is transferred to support booting microcontroller 38. Microcontroller 38 then enters the operational phase, in which (i) microcontroller 38 receives data transfer and programming from ECU 22, which is stored in memory 40 of microcontroller 38, and (ii) microcontroller 38 performs the scan of the tuning switches (Sp1-SpN, Se, S1, S2) in conditions appropriate for operation without stimulation and for operation during active stimulation, determines the appropriate combination for switches for each stage of operation, and places the respective configurations in registers 70 and 72 of group 58.

Microcontroller 38 also has a low power consumption sleep mode. While in sleep mode, microcontroller 38 can typically last up to half a second without power. The amount of time microcontroller 38 can last in sleep mode without power typically depends on conditions such as the power consumption of microcontroller 38 and of connected peripherals during sleep and how much energy can be stored locally. If power is not received by microcontroller 38 for longer than half a second, then microcontroller 38 shuts down and memory 40 is zeroed. Typically, at the start of a treatment ECU 22 transmits data and programming, e.g., parameters of the stimulation, to microcontroller 38. Microcontroller 38 stores this programming in memory 40 and uses it to configure the capacitor combinations stored in group 58 as well as time threshold 1 and time threshold 2 stored in timer logic unit 66. As long as microcontroller 38 only sleeps (as opposed to losing power for more than half a second and shutting down) then the programming from ECU 22 is stored and continues to run when microcontroller 38 wakes up from sleep mode. If however, microcontroller 38 shuts down and loses the data stored in memory 40, then microcontroller 38 is typically re-booted and re-programmed by ECU 22. In the event of microcontroller being re-booted, the start-up phase in which tuning logic state machine 46 tunes antenna circuit 26 is repeated.

As described hereinabove, for some applications, electrostimulator implant 20 includes ASIC 36′, which includes the logic state machine responsible for the initial tuning, e.g., tuning logic state machine 46, and microcontroller 38.

For some applications, in order to achieve the initial tuning, electrostimulator implant 20 includes non-volatile memory 78, e.g., a programmable read-only memory (PROM), in which a default parameter is stored. Tuning logic state machine 46 initially tunes antenna circuit 26 by initially setting the capacitance of antenna tuning circuit 30 based on the default parameter, e.g., logic circuitry 56 fetches an initial tuning value from non-volatile memory 78, e.g., one-time PROM register 78 (stored within group 58) and loads the initial tuning value into switches control register 54. For some applications, the default preconfigured parameter stored in non-volatile memory 78 is used for setting the capacitance of antenna tuning circuit 30 throughout the entire treatment (such as when utilizing tuning protocol A described hereinbelow).

For some applications (such as when utilizing tuning protocol B or C described hereinbelow), microcontroller 38 is able to tune antenna circuit 26 more accurately than is tuning logic state machine 46. This is because the initial tuning is based on a default value that provides a good enough power transfer for microcontroller 38 to be able to boot. However, other parameters, such as depth of implantation, may have an effect on the wireless power transfer between ECU 22 and electrostimulator implant 20, and the default parameter used for the initial tuning does not take such parameters into account. Once microcontroller 38 is in operational mode, microcontroller 38 performs the scan (as described hereinabove) of the tuning switches (Sp1-SpN, Se, S1, S2) in conditions appropriate for operation during the non-stimulation period and for operation during active stimulation for the specific conditions of the implant. For example, if electrostimulator implant 20 is implanted deeply within the tissue then microcontroller 38 typically finds the appropriate configurations for near maximum or maximum power transfer. However, in the case of a shallow implantation of electrostimulator implant 20, microcontroller 38 typically finds the appropriate configurations for lower power transfer.

Since there are deviations between different implants, having a single fixed value of initial tuning would not fit all implants. Therefore, during production a calibration of antenna tuning circuit 30 may be performed. In the production environment the power availability (and transfer) is not as strict as in the clinical settings. In particular, an external voltage supply may be used to power ASIC 36′ and microcontroller 38. Thus, during implant production the tunning may be tested and the default parameter for the initial tuning may be derived for each individual implant and stored in non-volatile one time memory 78 on ASIC 36′, to be used by tuning logic state machine 46. In this way, when tuning logic state machine 46 receives power and adjusts the initial tuning, the tuned antenna circuit 26 is able to provide enough power for microcontroller 38 to boot.

For some applications, ECU power unit 50 transmits the power to electrostimulator implant 20 at an initial high strength upon commencement of the start-up phase of operation of the electrostimulator implant, e.g., at a set near maximum or maximum strength that ECU 22 is able to transmit as a result of regulatory limits or due to ECU power consumption limits, or 70-95% of the near maximum or maximum strength capable, in order to provide sufficient energy to power tuning logic state machine 46 prior to tuning of the antenna circuit by the tuning logic state machine 46. Once the initial tuning is performed, ECU 22 subsequently reduces the strength of the transmitted power.

Utilizing various combinations of the features described hereinabove with respect to tuning logic state machine 46, three alternative examples of tuning protocols are provided as follows:

### Tuning Protocol A

1. Tuning logic state machine 46 uses PROM 78 for an initial adjustment of tuning of antenna circuit 26, such that after this initial tuning microcontroller 38 receives enough power to boot.
2. Tuning logic state machine 46 operates using the default parameter stored in PROM register 78 for setting the capacitance of antenna tuning circuit 30 throughout the treatment.

### Tuning Protocol B

1. Tuning logic state machine 46 uses PROM 78 for an initial adjustment of the tuning of antenna circuit 26, such that after this initial tuning microcontroller 38 receives enough power to boot.
2. Microcontroller 38 boots and scans possible tuning configurations in order to obtain optimized conditions (e.g., optimizing for power transfer or for reduced power transfer).
3. Microcontroller decides on a configuration for the switches (Sp1-SpN, Se, S1, S2) that is a compromise between (a) a configuration that would provide optimal adjustment of the antenna resonance for non-stimulation and (b) a configuration that would provide optimal adjustment of the antenna resonance for active stimulation, and writes the respective value in switches control register 54.
4. Tuning logic state machine 46 operates using the value as written in switches control register 54.

### Tuning Protocol C

1. Tuning logic state machine 46 uses PROM 78 for an initial adjustment of the tuning of antenna circuit 26, such that after this initial tuning microcontroller 38 receives enough power to boot.
2. Microcontroller 38 boots and scans possible tuning configurations in order to obtain optimized conditions (e.g., optimizing for power transfer or for reduced power transfer).
3. Microcontroller 38 then checks for an optimized configuration of the switches (Sp1-SpN, Se, S1, S2) for active stimulation and for non-stimulation and writes the respective values in active stimulation register 72 and non-stimulation register 70.
4. Tuning logic state machine 46 operates using the values as written in active stimulation register 72 and non-stimulation register 70. Logic state machine 46 switches between the two configurations based on signals from timer logic unit 66.

### Electrode Driver

Reference is now made to FIG. 5, which is a block diagram of electrostimulator implant 20, in accordance with some applications of the present invention. Neurostimulation pulses 80 are provided by electrode driver 45. Electrode driver 45 is connected to electrodes 24 (including electrode 1 and electrode 2) and controls the current injection into the tissue. In some case an external DC blocking capacitor 64 is connected in series to the tissue. Electrode driver 45 comprises two adjustable amplitude current sources 82, and a set of switches (P1, G1, P2, G2) that connects current sources 82 during the neurostimulation. For example, to provide an active stimulation current from electrode 1 to electrode 2, the switches P1 and G2 are closed while P2 and G1 are open. During this active stimulation period, DC blocking capacitor 64 is charged. When G1 and G2 are closed and P1 and P2 are open, DC blocking capacitor is discharged through the tissue, providing a DC balanced bi-phasic stimulation pulse. In order to provide the active stimulation current having the opposite polarity (from electrode 2 to electrode 1) the switches P2 and G1 are closed and P1 and G2 kept open. Opening all switches (P1, G1, P2, G2) disconnects current sources 82 from electrodes 24, thus eliminating the flow of current (e.g. during interphase between active stimulation and passive discharge). Finally, if P1 and G1 are closed, the current from the upper current source 82′ flows within ASIC 36′, creating a dummy stimulation (and similarly in the other polarity if P2 and G2 are closed), i.e., enabling the current to flow within the implant (e.g., for measurement purposes prior to the start of treatment) without injection of the current into the tissue.

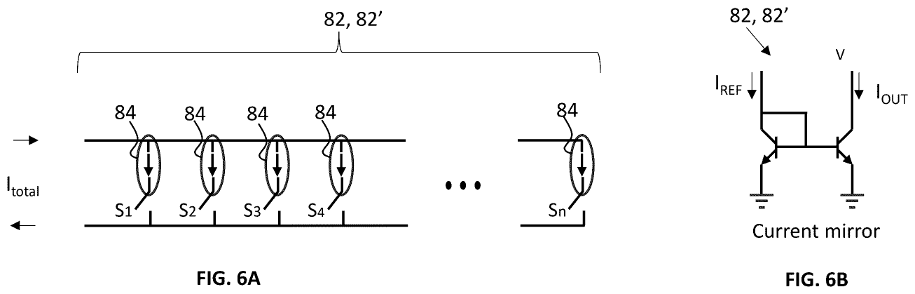

Reference is now made to FIG. 6A, which depicts details of current sources 82, in accordance with some applications of the present invention. Each of the two current sources 82 may comprise a plurality of separately-activatable current sources 84, each capable of providing minute currents, that are connected to provide the required current Iₜₒₜₐₗ. All of separately-activatable current sources 84 are able to provide the same current level and they are respectively connected or disconnected via switches S1 to Sn in order to enable adjustment of the total current provided by current source 82, 82′. For 8-bit resolution, 256 separately-activatable sources 84 are provided and 256 respective switches are controlled. The current amplitude of each of separately-activatable current sources 84 may be controlled by a single reference signal I_{REF} common to all separately-activatable sources (“reference current”).

Reference is now made to FIG. 6B, which shows an implementation of current sources 84, in accordance with some applications of the present invention. For some applications, the separately-activatable current sources may be implemented as a current mirror based digital to analog converter. This arrangement allows both a change of the full-scale current (by changing the reference current) as well as accurate digital discretization of the current amplitude (by controlling the amount of separately-activatable sources to be activated). In this arrangement, the biasing of the mirror transistor may be adjusted so it either operates as a mirror or disconnects the current flow. Other implementations may also be available, such as a binary weighted current mirror.

The reference current is provided by power unit 32, as shown in FIG. 5, and may be based on a bandgap voltage reference within power unit 32. A bandgap voltage reference provides a temperature independent voltage by circuitry comprising components having different temperature coefficients that tend to cancel each other. The exact voltage depends on the circuit design. For silicon electronics the voltage lies slightly above the bandgap of 1.17 eV.

Thus, for some applications, electrostimulator implant 20 includes:
one or more electrodes 24 (including electrode 1 and electrode 2);
antenna circuit 26, which comprises antenna receiving coil 28;
power unit 32, which comprises AC/DC conversion circuitry 34 configured to convert an AC voltage induced in antenna receiving coil 28 to one or more DC voltages; and
electrode driver 45, which (a) comprises current source 82 comprising a plurality of separately-activatable current sources 84 that are arranged to provide a combined current I_{TOTAL}, and (b) is configured to activate one or more of the plurality of separately-activatable current sources 84 to drive the one or more electrodes 24 to apply electrical stimulation to tissue of the subject at an amplitude based on which of separately-activatable current sources 84 are activated.

For some applications, the plurality of separately-activatable current sources 84 are configured to provide respective currents at a same, common amplitude. For some applications, the plurality of separately-activatable current sources 84 are controlled by a single reference signal common to all the current sources. For some applications, electrode driver is configured to (i) set a full-scale level of the amplitude of the electrical stimulation by setting an amplitude of the reference signal, and (ii) fine-tune the level of the amplitude of the electrical stimulation by selecting which of separately-activatable current sources 84 are activated. For some applications, a single current source 82 may be used. Alternatively, as described hereinabove, two current sources 82 may be used, each comprising a plurality of separately-activatable current sources 84, allowing electrode driver 45 to drive the stimulation in two polarities.

### Stimulation Control Unit

Reference is again made to FIG. 5. For some applications, stimulation logic state machine 52 is configured by microcontroller 38, as indicated by arrow 126, which sets the required current amplitude, the pulse width, and the polarity of neurostimulation pulses 80. Once set, stimulation logic state machine 52 sets the current source reference and connects the different separately-activatable current sources 84 to provide the required amplitude following timed signals provided by timer logic unit 66 within tuning logic state machine 46, or by another timer unit within ASIC 36′. Stimulation logic state machine 52 also manages the switches configuration to provide the required pulse polarity to electrodes 24, as indicated by arrow 128. At the end of the active stimulation phase of neurostimulation pulse 80, stimulation logic state machine 52 places the switches in the configuration fit for the interphase period, and following that it adjusts the switches and adjusts the amplitude of the current sources for discharge conditions. It is noted that once microcontroller 38 finishes setting the parameters for stimulation logic state machine 52, microcontroller 38 does not need to be involved in the process of controlling electrode driver 45, i.e., only stimulation logic state machine 52 and a timer logic unit (e.g., timer logic unit 66 within tuning state logic machine 46, or timer logic unit 92 as described hereinbelow) are involved in the process of controlling electrode driver 45. Microcontroller 38 may set the parameters for stimulation logic state machine 52 every neurostimulation pulse 80, or once per treatment (which requires more complexity of stimulation logic state machine 52).

### Clock and Timing Units

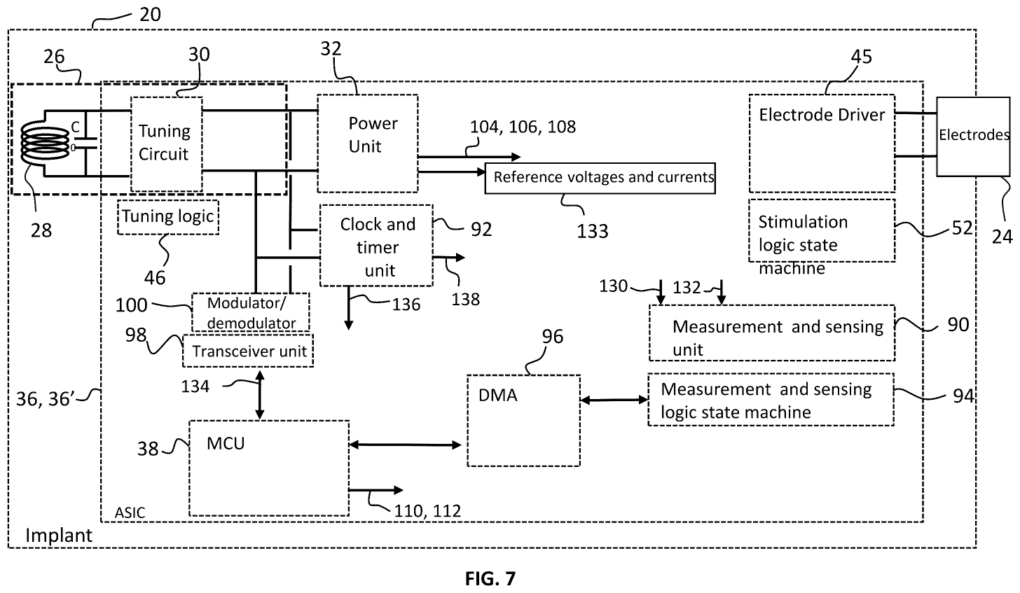

Reference is again made to FIG. 3. Clock 86 provides a clock signal (indicated by arrow 136 in FIG. 7) that is the basis of the system timing, i.e., for MCU 38 and the logic machines. In order that the timing will be synchronized with ECU 22, clock 86 may be derived from the wireless power carrier frequency coming from ECU 22 (e.g., by counting the phases of the RF signal). Alternatively, the clock signal may be derived from an external crystal or from an internal clock mechanism, such as a RC clock. These clocks, although not synchronized with the clock of the wearable ECU 22, may be used while the power signal is not detected in order to keep ASIC 36′ running. The switching between different clock sources is performed by a clock manager.

Timer units, e.g., timer logic unit 66 within tuning logic state machine 46, use the clock signal and counters to provide accurate timing signals (indicated by arrow 138 in FIG. 7) for the operation of other control units within ASIC 36′, e.g., the logic machines. Timed operations may include operations such as the timing of tuning logic state machine 46 as described hereinabove, the start of the active stimulation, the end of active stimulation, the start of the discharge, the timing of different measurements, the data transmission from the implant to the ECU, the transition to the sleep and others. For some applications, a clock and timer logic unit 92 may be disposed on ASIC 36′ outside of tuning logic state machine 46 (such as illustrated in FIG. 7) and may include any or all of the operations described hereinabove with respect to timer logic unit 66. The timer unit may detect specific features in the wireless power transfer to be used as a “zero-time event” for the timers. Such an event may be the start of the RF power transmission at each pulse, specific data modulation of the wearable device transmission, and others. The detection of these events requires that power unit 32 or the modulator/demodulator of the transceiver unit (described hereinbelow) be connected to the timer unit. This may be a built-in connection within ASIC 36′ to be detected by the timer unit, or an indirect connection via the microcontroller processing core 42.

### Measurement and Sensing

Reference is now made to FIG. 7, which is a block diagram of electrostimulator implant 20, in accordance with some applications of the present invention. A measurement and sensing unit 90 contains analog-to-digital converters and multiplexers enabling their connection to different sensing points within electrostimulator implant 20. Arrow 130 indicates the sensed signals, e.g., voltage, current, or temperature, received by measurement and sensing unit 90 from the different sensing points within electrostimulator implant 20. Arrow 132 represents internally-generated reference signals for the various sensed signals. These reference signals may be generated at least in part on ASIC 36′, as illustrated by power unit 32 generating reference voltages and currents in box 133. Some of the reference signals may be generated using internal electronics of electrostimulator implant 20 that are not on ASIC 36′. In addition, threshold detectors may be included for measuring signals, in which only detection of crossing a level is required rather than accurate measurement of the signal. For example, an overcurrent protection circuit may compare the measured current with a set threshold and provide a control signal if the current is above the set threshold. This signal may be used for aborting the electrode driver operation, or as an interrupt signal for the microcontroller to readjust the requested current. Threshold detectors may also be used for detecting if the DC voltage created by the power unit from the received RF power is high enough to enable the boot sequence of the microcontroller. Sensing signals may include the DC voltage created by electrostimulator implant 20 for its operation, the neurostimulation current, temperature sensing, the voltage drop across electrodes 24, and others.

### Measurement and Sensing Control Unit

A measurement and sensing logic state machine 94 manages measuring and sensing unit 90 in conjunction with timer unit 92. Microcontroller 38 sets measurement and sensing logic state machine 94 to perform measurements of different signals at different time points and store the measured data in a certain memory location. Following this setup, the measurement control unit adjusts the multiplexers and samples the signals on the proper timing and use a DMA (Direct Memory Access) unit 96 to store the information at the appropriate memory location, where it can be accessed by, for example, the microcontroller.

### Transceiver Unit

A transceiver unit 98 controls bidirectional data communication between electrostimulator implant 20 and ECU 22, as indicated by double-headed arrow 134. Transceiver unit 98 controls and configures a modulator/demodulator 100 and provides additional functions such as: encoding/decoding of the signal streams from/to modulator/demodulator 100, serialization/deserialization to transform the parallel multi-bit data interface of microcontroller 38 (and other digital units) into a series of sequential bits, CRC (cyclic redundancy check) or checksum capabilities, and encryption/decryption of messages for better security.

### Modulator/Demodulator

Modulator/demodulator 100 is an analog unit used for communicating information to and from the electrostimulator implant 20. Modulator/demodulator 100 is connected to antenna circuit 26. The demodulator translates changes of the power carrier amplitude or phase into digital signals that can be translated to bits. For example, the demodulator may measure the amplitude of the RF power signal and detect short time scale amplitude variations such as on-off keying. Alternatively, the demodulator may detect changes in the phase of the power carrier, thus enabling BPSK or QPSK data transmission. The modulator translates implant originated bit sequences into load modulation according to a protocol defined by the transceiver, based on the configuration set by the microcontroller.

For some applications, techniques and apparatus described herein are combined with techniques and apparatus described in one or more of the following applications, which are assigned to the assignee of the present application and incorporated herein by reference:
U.S. Pat. No. 8,755,893
U.S. Pat. No. 8,788,045
U.S. Pat. No. 9,186,504
U.S. Pat. No. 9,457,186
U.S. Pat. No. 9,597,521
U.S. Pat. No. 9,713,707
U.S. Pat. No. 9,764,146
U.S. Pat. No. 9,782,589
U.S. Pat. No. 9,861,812
U.S. Pat. No. 10,004,896
U.S. Pat. No. 10,105,540
U.S. Pat. No. 10,124,178
U.S. Pat. No. 10,653,888
U.S. Pat. No. 11,213,685
US Patent Application Publication 2018/0185631
US Patent Application Publication 2023/0122706
US Patent Application Publication 2023/0170138
PCT Publication WO 2011/154937
U.S. Provisional Application 63/434,699, filed Dec. 22, 2022

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-15. (canceled)

16. An electrostimulator implant, which is implantable in a subject for and use with an external transmitting coil, the electrostimulator implant comprising:
one or more electrodes;
an electrode driver, which is coupled electrically to the one or more electrodes;
an antenna circuit, which comprises: (i) an antenna receiving coil; and (ii) an antenna tuning circuit, which (a) is connected with the antenna receiving coil, (b) comprises a plurality of capacitors, at least some of which are switchable capacitors, and (c) is configured to have a variable capacitance that provides the antenna circuit with an adjustable resonance frequency, the capacitance varied by selective inclusion or exclusion of each of the switchable capacitors in the antenna tuning circuit;
a power unit, which comprises circuitry configured to convert an AC voltage induced in the antenna receiving coil to one or more DC voltages; and
an application-specific integrated circuit (ASIC), which (a) comprises a first portion of the antenna tuning circuit, which includes a first subset of the capacitors, and (b) is configured to:
tune the antenna circuit by setting the capacitance of the antenna circuit, and
control the electrode driver to drive the one or more electrodes to apply electrical stimulation to tissue of the subject, and
wherein a second portion of the antenna tuning circuit is off the ASIC, and includes a second subset of the capacitors, wherein the first and the second subsets are mutually exclusive.

17. The electrostimulator implant according to claim 16, wherein the first subset of the capacitors includes all of the switchable capacitors of the antenna tuning circuit.

18. The electrostimulator implant according to claim 16, wherein the first subset of the capacitors includes some of the switchable capacitors of the antenna tuning circuit, and wherein the second subset of the capacitors includes some of the switchable capacitors of the antenna tuning circuit.

19. The electrostimulator implant according to claim 16, wherein an average capacitance of the capacitors of the second subset is greater than an average capacitance of the capacitors of the first subset.

20. The electrostimulator implant according to claim 16, wherein the ASIC comprises a logic state machine, which is configured to tune the antenna circuit by setting the capacitance of the antenna tuning circuit.

21. The electrostimulator implant according to claim 16, wherein the ASIC comprises a microcontroller, which (a) comprises memory, a processor core, and input/output (I/O) ports, (b) is powered by the power unit, and (c) is configured to tune the antenna circuit by setting the capacitance of the antenna tuning circuit.

22. The electrostimulator implant according to claim 16, wherein the electrostimulator implant does not comprise a battery.

23. The electrostimulator implant according to claim 16, wherein the ASIC is configured to control the electrode driver to drive the one or more electrodes to apply every instance of the electrical stimulation to the tissue 0.1-5 ms after conversion of the AC voltage to the one or more DC voltages by the power unit.

24. An electrostimulator system comprising the electrostimulator implant of claim 16, the electrostimulator system further comprising an external control unit, which comprises the external transmitting coil and an external-control-unit power unit configured to transmit power to the electrostimulator implant by providing a current to the external transmitting coil.

25. The electrostimulator system according to claim 24, wherein the external-control-unit power unit is configured to drive the external transmitting coil at a single fixed frequency.

26-60. (canceled)
